(19) United States
(12) Patent Application Publication
(10) Pub. No.: US 2022/0160482 A1
(43) Pub. Date: May 26, 2022

(54) MIRROR APPARATUS FOR DENTAL EVACUATOR

(71) Applicant: SUCTION SOLUTIONS LLC, Ypsilanti, MI (US)

(72) Inventors: Michael D Scherer, Soulsbyville, CA (US); Jason Kluttz, Ypsilanti, MI (US)

(73) Assignee: SUCTION SOLUTIONS LLC, Ypsilanti, MI (US)

(21) Appl. No.: 17/100,641

(22) Filed: Nov. 20, 2020

(51) Int. Cl. A61C 17/06; A61C 17/08

(52) U.S. Cl. CPC A61C 17/088; A61C 17/096; A61C 17/08

(57) ABSTRACT

A removably attachable mirror apparatus is adapted to be attachable to a suctioning tool of a dental evacuation system for removal of aerosols during a dental procedure. The mirror apparatus includes a closed-loop collar having a mirror post retaining portion adapted to retain a dental mirror. The stem is typically bent at an end in proximity to the mirror to allow for better viewing of a patient's mouth. The collar is sized to create an interference fit over the suctioning tool but is removable after use. The collar may be made of a metal, resin, plastic or silicone. The collar may be 3-D printed. The mirror apparatus may be made to be autoclavable between uses. Alternatively, the collar may be made to be disposable after a single use. The mirror apparatus provides advantages of easy use, low cost and improved remediation of potentially dangerous aerosols generated during common dental procedures.

### FIELD OF THE INVENTION

The invention generally relates to dental mirrors and evacuation systems. More particularly, the invention relates to a mirror apparatus adapted to couple with a dental evacuation tool of a dental evacuation system.

### BACKGROUND

Dentistry requires working in close proximity to biological contaminants produced during dental procedures including teeth cleaning, cavity filling, and oral surgery. Contaminants include bacteria and viruses such as COVID-19, along with other blood or saliva-borne microbials, and solids. Dental practitioners including dentists, oral surgeons, and dental hygienists are at risk of exposure to biological contaminants that become aerosolized during common dental procedures. This is a result of using dentistry tools such as a dental drill or handpiece, and other high-frequency rotating or vibrating equipment that in use are predisposed to create a high volume of aerosols. Once aerosolized, it is well known that biological contaminants can linger in the air, thus creating a potential hazard to anyone in the aerosol's range of travel. Therefore, it would be beneficial to develop a system for removing, or at least substantially reducing, aerosols created during dental procedures.

Various aerosol remediation systems for dental application exist and include, but are not limited to, standalone vacuums having a vacuum inlet to be positioned near the patient's face. These systems tend to be bulky and expensive. It is not practical or cost effective to have one of these units in each treatment room at a dentist's office. Other solutions include, for example, a dental evacuation mirror disclosed in U.S. Pat. No. 7,553,158 which is also relatively costly and requires the purchase of proprietary equipment rather than making use of existing equipment at a dental office. Such systems may also restrict the inflow of aerosols into a vacuum system by restricting or distributing the positioning of vacuum inlets. Therefore, there is opportunity to provide a cost-effective alternative that works with existing dental office evacuation systems and does so without restricting the vacuum force which in turn limits the remediation of aerosolized biological contaminants.

Most, if not substantially all dental practices have an evacuation system used to aid the dentist in suctioning the patient's mouth during a dental procedure. Evacuation systems are used to help with suctioning of liquids such as saliva and blood, along with suctioning debris generated during a dental procedure. Inherently, dental evacuation systems create a vacuum in and around the patient's mouth thus removing aerosols. However, dentists have limited ability to hold multiple instruments including a dental procedure tool such as a handpiece and multiple other instruments such as a mirror and/or light. Further, it cannot be assumed that all dentists have an assistant nearby to hold extra instruments during a procedure to permit “four-handed dentistry.” In addition, there is limited space in a patient's mouth to accommodate multiple instruments. Therefore, there is a need to provide an ability to remove aerosols generated by dental procedures without interfering with the dental procedure.

Preferably an aerosol removal system would take advantage of a dentist's existing investment in dentistry equipment including an existing evacuation system commonly found in dental offices. Further, use of an existing evacuation system would be best accomplished by not limiting or occluding the vacuum force created by existing suctioning equipment. Cost savings and convenience are needed to facilitate the important job of removing aerosols, including the contaminants in dental procedure created aerosols. In addition, combining multiple instruments such as a mirror and evacuation system creates a desirable synergistic solution.

### SUMMARY OF THE INVENTION

The present embodiments comprise a mirror apparatus adapted to couple to a suctioning instrument of an existing dental evacuation system. The mirror apparatus comprises a closed-loop collar adapted to forcibly slide onto a suctioning instrument or tool such as a straw of a dental evacuation system. An outer portion of the collar is coupled to a stem in the form of a rod, wherein the stem at an opposite end is bent at an angle. The angled end is coupled to a mirror holder wherein the mirror holder holds a mirror. In a nonlimiting embodiment, the mirror is fixed at an of angle of 45° or 22.5° relative to the mirror stem. In a nonlimiting embodiment the bent portion of the mirror stem is a hinge, thus allowing for adjustment of the mirror angle relative to the stem.

The collar has an inside opening that is slightly larger than the outside diameter of the suctioning instrument wherein the collar is adapted to be frictionally and releasably coupled to the suctioning instrument. The mirror apparatus is adapted to be removably slidable onto the suctioning instrument where it is held in substantially fixed position during a dental procedure. The mirror apparatus is adapted to be reusable including after sterilizing in an autoclave. In an embodiment the collar is substantially ring-shaped. In a nonlimiting embodiment, the internal diameter of the collar is 0.439 inches+−0.002 inches (11.150 mm+−0.02 mm).

In a nonlimiting embodiment the collar is a ring with an internal protrusion adapted to interface with the straw thus impeding movement of the collar on the straw and limiting unintentional removal of the device from the straw. In a nonlimiting embodiment the ends of the rings have a circumferentially chamfered edge that will aid in putting the ring onto the dental tool or straw. In a nonlimiting embodiment the collar is made from aluminum. In a nonlimiting embodiment the aluminum is anodized, and color coded to correspond to a mirror size.

In a nonlimiting embodiment the collar is made from at least one of stainless steel, copper and metal alloy.

In another nonlimiting embodiment the collar is made from at least one of a resin, plastic, nylon or silicone. In a nonlimiting embodiment the collar is disposable. In this embodiment, the collar is adapted to releasably receive the stem of the mirror which can be put into a receptor portion of the collar. In this embodiment the mirror can be removed so that it can be autoclaved and the collar disposed of In a nonlimiting embodiment the mirror stem the mirror stem has a notch adapted to connect to a notch receptor within the collar. In another nonlimiting embodiment the mirror stem includes threads that allow for screwing the mirror assembly into the stem receptor of the collar.

In another nonlimiting embodiment the mirror apparatus comprises at least a first collar coupled to a second collar wherein the first and second collar are adapted to slide onto the straw.

In yet another nonlimiting embodiment the collar, or collars are 3-D printed. In a 3-D printed embodiment a receptor area in the form of hole is left for inserting the stem of a mirror into the hole. In this embodiment the collar of the mirror apparatus is made from a suitable medical and/or dental grade resin.

The present embodiments comprise an adaptable mirror apparatus that provides a dental practitioner with a convenient, easy to use, cost effective system that will easily work with a plurality of dental office evacuation systems for remediation of biological contaminants including contaminants in aerosols.

### BRIEF DESCRIPTION OF THE DRAWINGS

Advantages of the invention will be readily appreciated as the same becomes better understood by reference to the following detailed description when considered in connection with the accompanying drawings, wherein:

FIG. 1 is a perspective view of a nonlimiting embodiment of a mirror apparatus of the invention in the environment of a prior art dental evacuation system shown in broken lines;

FIG. 2 is a side perspective view of the mirror apparatus of FIG. 1;

FIG. 3 is back perspective view of the mirror apparatus of FIG. 2;

FIG. 4 is a cross-sectional view of the mirror apparatus along the line 4-4 of FIG. 3;

FIG. 5 is a cross-sectional view of the mirror apparatus along the line 5-5 in FIG. 3;

FIG. 6 is a side perspective view of an alternative embodiment of the mirror apparatus of the invention;

FIG. 7 is a top view of a base of the alternative embodiment of the mirror apparatus of FIG. 6;

FIG. 8 is cross-sectional view along the line 8-8 of FIG. 7;

FIG. 9 is perspective view of an alternative embodiment of the mirror apparatus of the invention as seen in the environment of a prior art dental evacuation system shown in dotted or broken lines;

FIG. 10 is a perspective view of a base of the mirror apparatus seen in FIG. 9;

FIG. 11 is a cross-sectional view along the line 11-11 of FIG. 10;

FIG. 12 is a perspective view of mirror apparatus of FIG. 9, shown in spaced apart separation of the base and mirror assembly;

FIG. 13 is a perspective view of the mirror apparatus of FIG. 9, shown in spaced apart separation of the base and an alternative embodiment of the mirror assembly; and

FIG. 14 is perspective view of the mirror apparatus of FIG. 9 seen in the environment of being used, including a prior art patient, dental practitioner, dental tool, and evacuation system as shown in broken lines.

### DETAILED DESCRIPTION OF THE DRAWINGS

The subject embodiments of the invention relate to a device including a mirror apparatus adapted to attach to a dental evacuator thus allowing for removal of aerosols while performing common dental procedures. The subject system provides numerous benefits including an ability to easily integrate with existing equipment commonly found in a dentist's office. The device is compact, light weight and inexpensive when compared to the alternative of buying specialized equipment needed for removal of aerosols. Importantly, the device allows for improved aerosol removal by keeping the device including the mirror near to the work area and not reducing the vacuum force of the dental evacuator.

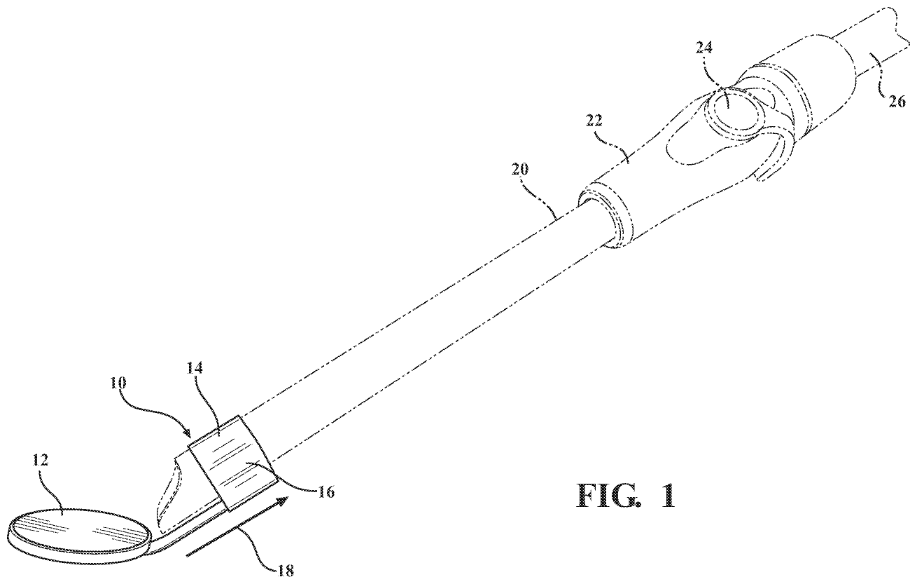

More particularly, with reference to FIG. 1 the subject embodiments of the mirror apparatus 10 of the invention comprise a mirror assembly 12 and a base 14 wherein the base 14 comprises at least one closed-loop collar 16 adapted to slide in a rectilinear direction 18 onto a suctioning tool 20 of an evacuation system (not shown). The suctioning tool 20 is sometimes referred to as a “straw” and is commonly a disposable plastic tube used to introduce suction into a patient's mouth during a dental procedure. The suctioning tool 20 may come in various shapes, sizes, and lengths, and it is an intent of the invention to be adaptable to different suctioning tool shapes, sizes and lengths. The suctioning tool 20 is coupled to a suctioning device 22 having a valve 24 for adjusting and turning on/off a suctioning force. The suctioning device 22 is connected to a hose 26 which in turn is connected to the suctioning system included in the dental evacuation system. It should be appreciated that the invention's intended use is not limited to one type or style of dental suctioning tool 20, suctioning device 22 or evacuation system. It should also be understood that all references to the suctioning tool 20, suctioning device 22, evacuation system, or a dental patient are for environmental purposes of describing the invention and are not intended to be a claimed element or otherwise a part of the invention disclosed herein.

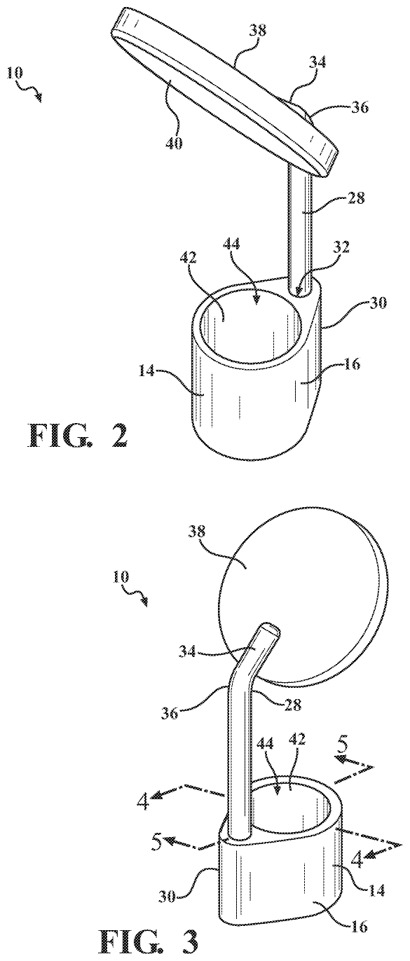

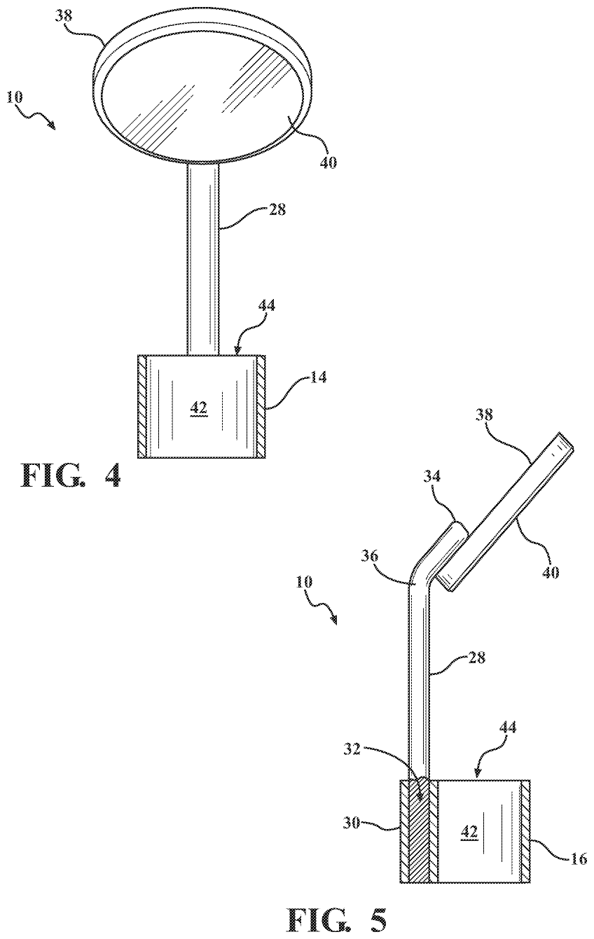

With added reference to FIGS. 2-5, the mirror apparatus 10 includes a mirror stem 28 retained in an opening 32 of a mirror post retaining portion 30 of the base 14. The mirror stem 28 can be fixated in the mirror post retaining portion 30 by means of mechanical fixation such as screws (not shown) going through the mirror post retaining portion 30 of the base 14 and into the mirror stem 28, or by use of an adhesive. Alternatively, the mirror stem 28 can be press fit into the opening 32 of the mirror post retaining portion 30, thereby permanently joining the two together. As another alternative, the mirror stem 28 can be threaded into a corresponding threaded opening 32 of the retaining portion 30 thereby allowing for a threaded coupling of the mirror stem 28 and the base 14. This embodiment is particularly suitable for using existing mirror assemblies that are available with threading already on a portion of the mirror stem. This embodiment allows for use of existing tapered mirror stems or simple (i.e. straight) mirror stems with threading.

At a distal end 34, the mirror stem 28 is bent at an angle relative to the stem 28 portion disposed in the base 14. A transition portion 36 of the stem 28 is coupled to the mirror housing 38 which holds a mirror 40. The transition portion 36 can be a bend in the stem 28, typically of 45° or 22.5° relative to the stem 28 thus allowing for the mirror 40 to offer better views of a patient's mouth during dental procedures. Other angles of inclination between the mirror and mirror stem are anticipated.

In an embodiment, the transition portion 36 is adjustable (not shown) to allow the user to change the inclination of the mirror 40 relative to the stem 28. This can be accomplished in a manner well known to those skilled in the art and includes a hinge joint. The mirror housing 38 and mirror 40 are typically round but could also be other shapes such as oval, square, or rectangular.

The closed-loop collar 16 forms a circumferential wall 42 that forms a tool receptor opening 44 adapted to receive and releasably couple with the suctioning tool 20. The circumferential wall 42 is circular in shape. However, in nonlimiting embodiments the circumferential wall 42 may be formed into other shapes such as an oval, square, or rectangle and may correspond to the shape of the suctioning tool 20 which may also be oval, square, rectangular or other shape. In a nonlimiting embodiment, the collar 16 of the mirror apparatus 10 can be color coded to correspond to different sizes of the mirror housing 38 and mirror 40. This allows a dental practitioner to easily determine the size of the mirror 40 needed for a particular dental procedure.

The tool receptor opening 44 is sized to correspond to the size of the suctioning tool 20 to be coupled with. In a nonlimiting embodiment the diameter of the tool receptor opening 44 is 0.439 inches or 11.150 mm to couple with the suctioning tool 20, such as a straw with an exterior diameter of ˜0.438 inches. In this embodiment the inside diameter of the tool receptor opening 44 is only slightly larger than the outside diameter of the suctioning tool 20 which allows the mirror apparatus 10 to be urged onto the suctioning tool 20 prior to use. It should be appreciated that the size of the tool receptor opening 44 and the suctioning tool 20 may vary. It should also be understood that the suctioning tool 20 may be oval or some other non-circular shape.

The close association in sizes between the tool receptor opening 44 and the suctioning tool 20 may be characterized as a frictional retention of the mirror apparatus 10 on the suctioning tool 20. After being urged onto suctioning tool 20, the mirror apparatus 10 is frictionally held in place on the suctioning tool 20 and not easily removable. In this regard the mirror apparatus 10 is safe from becoming easily dislodged during normal dental procedures. The length of the collar 16 also assists in offering stability from easy dislodging of the mirror apparatus 10 from the suctioning tool 20. A length of the collar 16 of less than 0.5 inches (˜1 cm) has proven sufficient.

The base 14 is made from at least one of aluminum, stainless steel, copper, and metal alloy. The base 14 may be made by extruding or milling aluminum. In this embodiment the aluminum may be anodized. In another nonlimiting embodiment the base 14 is made from at least one of a resin, plastic, nylon or silicone. The base 14 may be molded or 3-D printed.

With added reference to FIGS. 6-8, a nonlimiting embodiment of the invention 10 includes a restrictive collar 46 with a protruding portion 48 in the circumferential wall of the tool receptor opening 44. The protruding portion 48 somewhat occludes the tool receptor opening 44 thus further restricting the movement, particularly rotational movement of the closed-loop collar 16 on the straw. The protruding portion 48 is represented as a rectangular portion of the circumferential wall 42 but could be any number of different shapes that will offer the advantage of restricting movement of collar 16 on the suctioning tool 20. For example, the protruding portion 48 could be a protrusion in only a portion of the circumferential wall 42. In another form, the protruding portion 48 could be circumferential protrusion of the circumferential wall 42. The protruding portion 48 will restrict movement of the collar 16 in both a rectilinear direction relative to the suctioning tool 20 and rotationally around the suctioning tool 20. Therefore, this embodiment further protects against unintentional removal of the device from the suctioning tool 20.

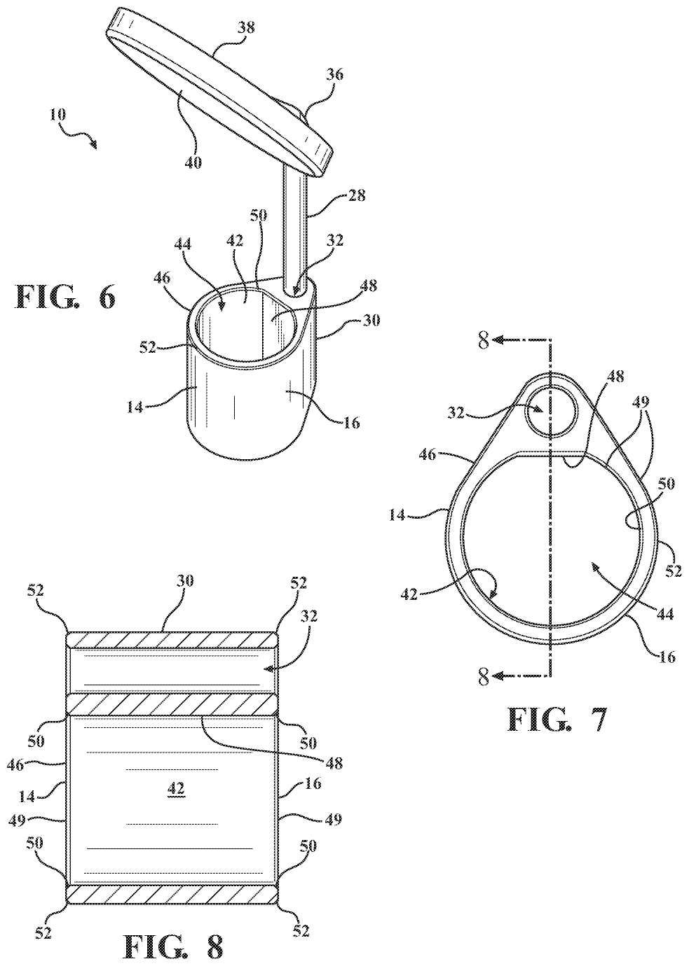

With particular reference to FIGS. 7 and 8, in a nonlimiting embodiment, the edges 49 of the base 14 have a chamfered inner surface 50 and a chamfered outer surface 52. The chamfered inner surface 50 makes it easier to urge the base 14 onto the suctioning tool 20 of a dental evacuation system. The chamfered outer surface 52 will help reduce the potential for a patent's mouth including his/her teeth from bumping against an edge that would exist if the chamfering was not present. It should be appreciated that the base 14, could, in an embodiments have either one of the chamfered inner surface 50, or the chamfered outer surface 52.

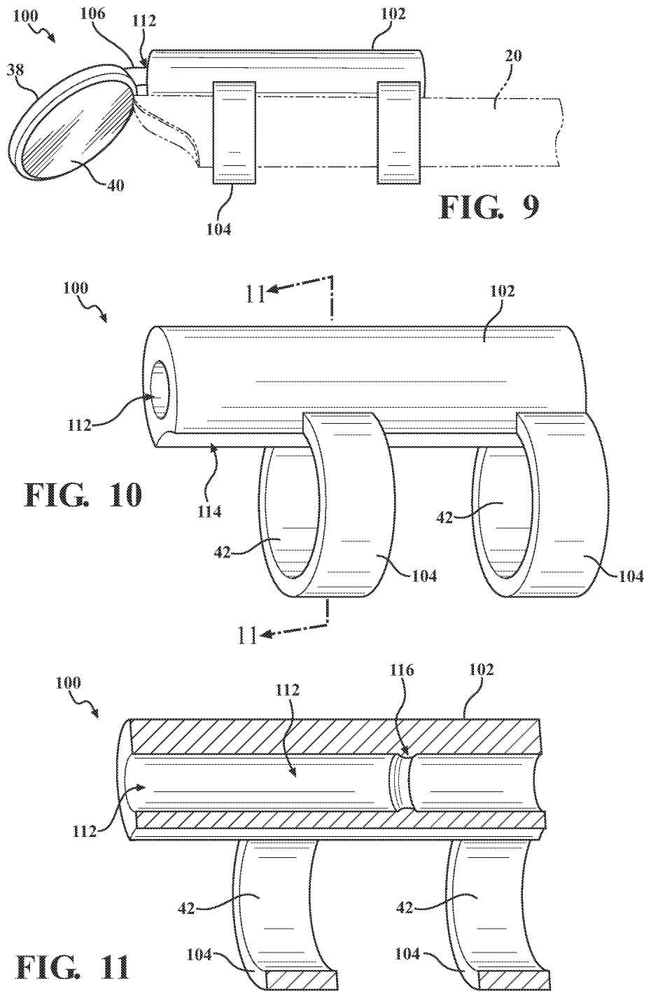

Referring to FIGS. 9-13, in a nonlimiting embodiment, the mirror apparatus 100 of the invention includes an extended base 102 and at least two closed-loop collars 104. This embodiment is preferably made from a resin, including resins specifically intended for medical and dental applications. Examples of resins include NextDent SG and SprintRay Surgical Guide 2, which can be heat sterilized for reuse, and others that are intended for single use only, like NextDent Orto MT and SprintRay IDB. This embodiment is also suitable for being 3-D printed. The at least two collars 104 are sized to closely fit over the suctioning tool 20 creating a releasably frictional coupling.

The mirror assembly 106 includes an elongated mirror stem 108 that is inserted in a rectilinear direction 110 into a mirror stem retaining portion 112 of the extended base 102. The extended base 102 includes a recessed portion 114 that conforms to the radial curvature of the suctioning tool 20.

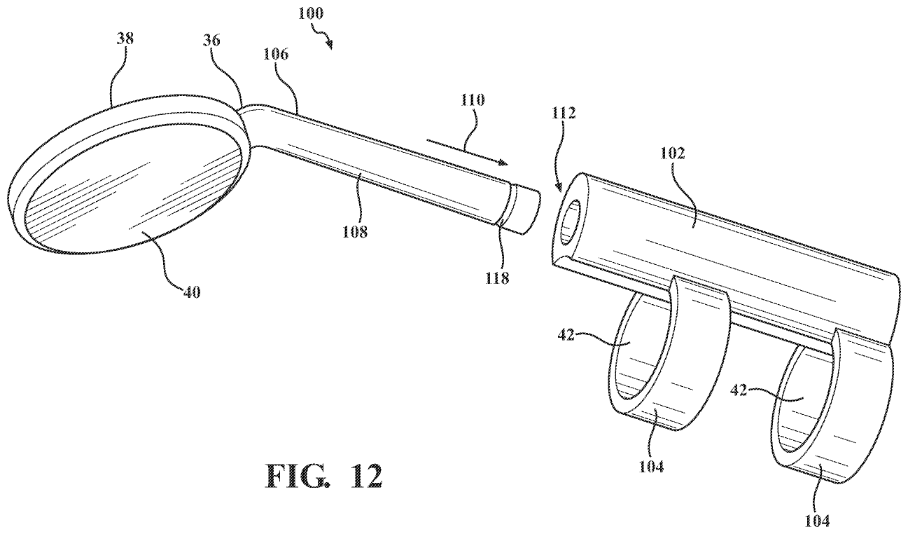

The mirror stem retaining portion 112 includes an indentation 116 that in a first embodiment (FIG. 12) corresponds to a notch 118 in the elongated mirror stem 108. In preparation for use, the mirror stem 108 is inserted into the mirror stem retaining portion 112 in the rectilinear direction 110 until the notch 118 couples in a snap-fit type manner with indentation 116. Thus, the mirror assembly 106 is locked into place for using the mirror apparatus 100 for a dental procedure.

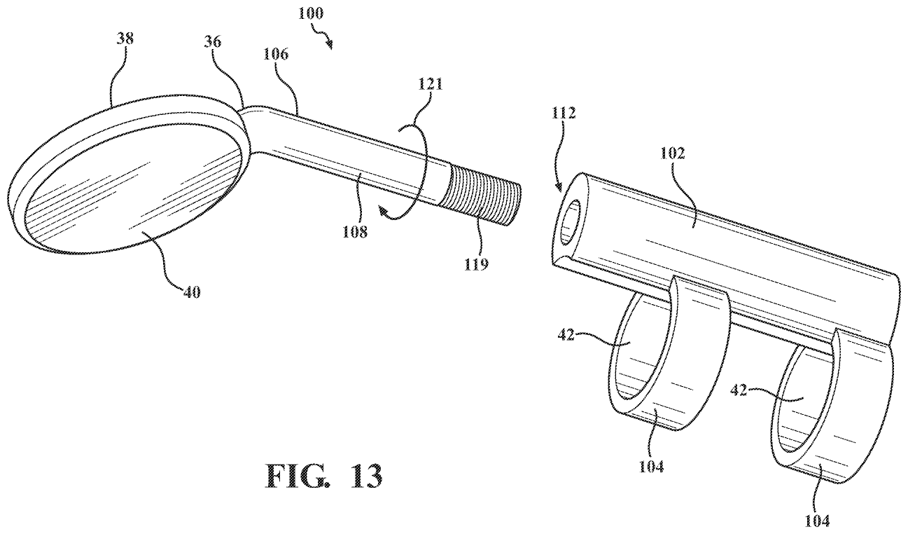

With particular reference to FIG. 13, in an alternative embodiment, the mirror stem 108 has threads 119 in place of the notch 118. In this alternative embodiment, the threaded portion of the mirror stem 108 is rotated in a standard clockwise rotational direction 121 while being inserted into the mirror stem retaining portion 112. Pressing the threads 119 of the mirror stem 108 against the indentations 116 while rotating the mirror assembly 106 forms a counter-threading of the indentations 116 thus joining the two together and preventing the mirror assembly 106 from dislodging. This embodiment is particularly suitable for using existing mirror assemblies having a threaded stem.

After use, the mirror assembly 106 can be retracted from the extended base 102 thereby allowing for better sanitizing including autoclaving of the parts. In an embodiment, the extended base of the mirror apparatus 100 is intended for a single use and then is disposable, leaving only the mirror assembly 106 to be cleaned. The apparatus of the invention allows dentists, hygienists, and dental assistants to minimize aerosols while working clinically.

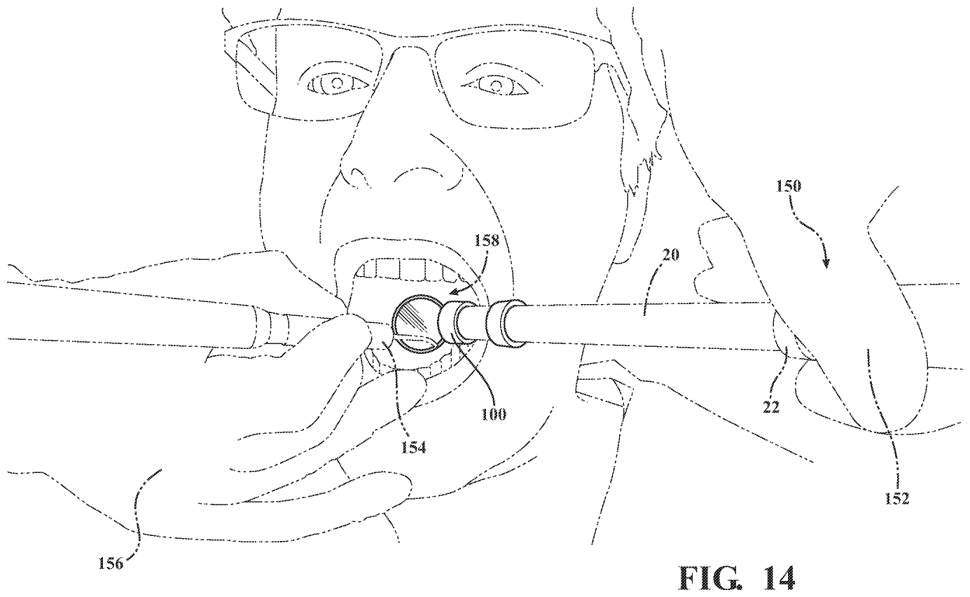

With added reference to FIG. 14, in use the mirror apparatus 100 is first placed on the suctioning tool 20 of the suctioning device 22 of an evacuation system (not shown), as explained with reference to FIG. 1. Suction is activated in the suctioning tool 20. Next, the dental practitioner 150 holds the suctioning device 22 in a first hand 152, typically non-dominant hand, and holds a separate dental tool 154 in the dominant hand 156 where the dental tool is one prone to create aerosols through use during a dental procedure. The mirror apparatus 100 is introduced into the mouth 158 of the patent. Importantly, the mirror apparatus 100 is easily positioned in immediate proximity to the dental procedure's area of focus where most aerosols are being generated. The mirror of the mirror apparatus allows for easy viewing and illumination of the area of focus during the dental procedure.

When the dental treatment is completed the dental practitioner simply removes the mirror apparatus from the straw by pulling it off by hand. In most embodiments, the mirror apparatus can be autoclaved with the mirror attached to the base. Alternatively, in embodiments where the mirror assembly is intended to be removable from the base it is removed for autoclaving. In other embodiments as already described, the mirror assembly is first removed for autoclaving and then the base is disposed of. In all embodiments, the mirror apparatus provides an inexpensive, convenient and practical way to alleviate aerosols generated during common dental procedures.

The invention has been described in an illustrative manner. It is to be understood that the terminology, which has been used, is intended to be in the nature of words of description rather than of limitation. Many modifications and variations of the invention are possible in light of the above teachings. Therefore, within the scope of the appended claims, the invention may be practiced other than as specifically described.

1. A mirror apparatus adapted to attach to a dental suctioning tool for removal of aerosols during dental procedures, the mirror apparatus comprising:
a base adapted to couple with a mirror assembly, wherein the mirror assembly includes a mirror stem and a mirror, wherein the base has a mirror retainer opening adapted to receive the mirror stem and retain the mirror stem; and
a collar forming a closed-loop, wherein the collar is disposed in the base, wherein the collar has an opening of a defined and fixed size adapted to allow the collar to slide onto a suctioning tool and remain frictionally retained on the suctioning until forcibly removed.

2. The mirror apparatus of claim 1, wherein more than one collar is disposed in the base.

3. The mirror apparatus of claim 1, wherein the base has a chamfered edge around the opening.

4. The mirror apparatus of claim 1, wherein the stem of the mirror is threaded and rotationally culpable with the base.

5. The mirror apparatus of claim 1, wherein the base is made out of at least one of steel, copper, aluminum, resin and a metal alloy.

6. The mirror apparatus of claim 1, wherein the base is made out of a resin and the base comprises a ring-shaped annular reduced portion of the mirror retainer opening, wherein the mirror stem has threading and is adapted to rotatably couple with the ring-shaped annular reduced portion.

7. The mirror apparatus of claim 6, wherein the base is 3-D printed.

8. The mirror apparatus of claim 1, wherein the base is made out of a resin and the base comprises a ring-shaped annular reduced portion of the mirror retainer opening, wherein the mirror stem has annular notch adapted to mate with the ring-shaped annular reduced portion.

9. The mirror apparatus of claim 8, wherein the mirror stem snap-fits into a retained position in the mirror retainer opening.

10. The mirror apparatus of claim 1, wherein the collar includes a protruding portion that protrudes into an otherwise circular ring.

11. The mirror apparatus of claim 1, wherein the base is color coded to correspond to a mirror size.

12. The mirror apparatus of claim 1, wherein the mirror stem comprises a hinge in proximity to the mirror, wherein the angle of the mirror can be adjusted relative to the mirror stem.

13. An apparatus adapted to attach to a dental suctioning tool for removal of aerosols during dental procedures, the apparatus comprising:
a base adapted to couple with a mirror assembly, wherein the base comprises a mirror retainer opening adapted to receive a stem of the mirror assembly and releasably retain the mirror assembly in the base; and
a collar forming a closed-loop, wherein the collar is disposed in the base, wherein the collar has an opening of a defined and fixed size adapted to allow the collar to slide onto a suctioning tool and remain retained on the suctioning until forcibly removed.

14. The apparatus of claim 13, further comprising:
a mirror assembly including the stem and a mirror coupled to an end of the stem.

15. The mirror assembly of claim 14, wherein the stem includes a threaded portion, wherein the mirror retaining portion is adapted to receive and be retained by the threaded portion.

16. The apparatus of claim 13, wherein the base comprises a plurality of collars.

17. The apparatus of claim 13, wherein the base is made out of at least one of a metal, metal alloy and resin.

18. The apparatus of claim 13, wherein the base is 3-D printed.

19. The apparatus of claim 14, wherein the stem includes a hinge in proximity to the mirror and is adapted to adjust the angle of the mirror relative to the stem.

20. A mirror apparatus adapted to attach to a dental suctioning tool for removal of aerosols during dental procedures, the mirror apparatus comprising:
a base coupled to a mirror stem, wherein the mirror stem is coupled to a mirror assembly including a mirror; and
a collar forming a closed-loop, wherein the collar is disposed in the base, wherein the collar has a circular opening of a defined and fixed size adapted to allow the collar to slide onto a suctioning tool and remain retained on the suctioning until forcibly removed.
